(19) United States
(12) Patent Application Publication
(10) Pub. No.: US 2026/0220601 A1
(43) Pub. Date: Jul. 30, 2026

(54) SYSTEM AND METHOD FOR MANAGING HEALTHCARE RECORDS

(71) Applicants: Danielle McCord, Merrick, NY (US); JIMMY WAGNER, BROOKLYN, NY (US)

(72) Inventors: Danielle McCord, Merrick, NY (US); JIMMY WAGNER, BROOKLYN, NY (US)

(21) Appl. No.: 19/439,962

(22) Filed: Jan. 5, 2026

Related U.S. Application Data
(60) Provisional application No. 63/749,714, filed on Jan. 27, 2025.

(51) Int. Cl. G06Q 10/10; G16H 10/60

(52) U.S. Cl. CPC G06Q 10/10; G16H 10/60

(57) ABSTRACT

A system and method for managing healthcare data that provides a cost-navigation platform that allows patients, healthcare providers, and insurance entities to obtain accurate, real-time estimates of medical and dental treatment costs. The system includes a processor and a memory storing executable modules comprising an interface module, a data module, an analysis module, and an output module. The data module automatically retrieves insurance, healthcare provider, and patient information from authenticated databases through secure communication protocols. The analysis module computes cost estimates based on provider fee schedules, policy terms, deductibles, co-pays, and plan rules. The output module displays an explainable cost estimate range and detailed breakdown through a user interface. Artificial intelligence continuously refines estimation accuracy using historical claim and payment data. The disclosed system thereby enables transparent, up-to-date, and patient-specific cost assessment for healthcare services.

### CROSS REFERENCE TO RELATED APPLICATIONS

This application claims priority from a U.S. Provisional Patent Appl. No. 63/749,714, filed on 01/27/2025, which is incorporated herein by reference in its entirety.

### FIELD OF INVENTION

The present invention relates generally to systems and methods for managing healthcare data. More particularly, the present invention pertains to a health and dental cost navigation system designed for use by patients and healthcare professionals to estimate and analyze the cost estimates for healthcare services.

### BACKGROUND

Healthcare services involve a variety of costs that depend on the type of treatment, facilities used, and duration of care. Numerous factors influence the final cost of healthcare services. For treatments that span multiple days, different charges continue to accumulate daily. Patients receiving such healthcare services often find it difficult to monitor and estimate the total cost, especially while focusing on their treatment and recovery.

The complexity further increases due to the involvement of healthcare insurance companies, which maintain distinct contractual arrangements with healthcare service providers. Under these contracts, insurance companies apply different contribution scales for various medical and dental procedures. Each procedure is assigned a standardized medical code, and insurers pay a specific percentage of the associated cost. Patients are required to pay the remaining balance as part of their co-pay. However, each insurance plan varies in its coverage percentages, limitations, and downgrades applied to specific procedures.

Consequently, patients and healthcare providers often remain uncertain about the patient’s financial responsibility for a given treatment. Patients requiring significant medical or dental procedures frequently experience long delays before receiving final cost estimates, which may arrive too late for timely decision-making.

Existing systems for tracking healthcare costs typically manage the healthcare data and bills, and the same cannot be accurately used to estimate the cost of similar treatment. This lack of clarity adversely impacts patients’ ability to access appropriate healthcare, creates unnecessary mental stress, and consumes substantial time for both patients and healthcare staff—particularly when same-day treatment is desired, but cost details are unclear.

Accordingly, there exists a need for a system and method that can overcome the foregoing challenges by providing a comprehensive and efficient way to manage, estimate, and navigate healthcare and dental costs for patients and professionals alike.

### SUMMARY OF THE INVENTION

The following provides a simplified summary of certain embodiments of the present invention to facilitate a basic understanding of its features and advantages. This summary is not intended to be an exhaustive overview of all contemplated embodiments or to define the full scope of the invention. Rather, it introduces key concepts that are described in greater detail in the subsequent sections.

The principal object of the present invention is directed to a system and method for managing healthcare costs in an efficient and transparent manner.

Another object of the present invention is to enable rapid estimation of healthcare costs prior to treatment.

Still another object of the present invention is to provide automation of cost estimation with minimal human intervention.

Yet another object of the present invention is to facilitate same-day treatment by enabling insurance providers to quickly confirm cost estimates.

A further object of the present invention is to reduce accounting overhead, administrative delays, and manual errors associated with healthcare billing.

Yet a further object of the present invention is to eliminate uncertainty for patients, healthcare professionals, and insurance companies by simplifying the interpretation of complex contribution scales.

An additional object of the present invention is to provide accurate and automated cost estimates even during emergencies such as pandemics or endemics, when healthcare staff may be limited and patient volume high.

A further object of the present invention is to prevent treatment delays caused by slow or unclear cost determination.

Another object of the present invention is to enable patients and healthcare providers to make informed financial and medical decisions.

Still another object of the present invention is to bring transparency to the cost estimation process in healthcare and dental services.

A further object of the present invention is to allow patients, doctors, medical facilities, and other professionals to obtain fair, real-time, and accurate estimates of treatment costs.

In one aspect, disclosed is a system and method for automated and intelligent cost navigation that integrates multi-source data to generate precise and dynamic healthcare cost estimates. The system can receive and process insurance provider contracts, coverage policies, and healthcare institution fee schedules, along with patient-specific insurance parameters. Based on user input identifying a desired healthcare or dental service, the system applies relevant deductibles, co-pays, and coverage percentages to determine an accurate cost estimate.

In certain embodiments, the system can also calculate and present a cost range, including minimum and maximum possible expenses, rather than a single value, to ensure transparency and minimize post-treatment discrepancies for both patients and service providers.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing an environment of the system, according to an exemplary embodiment of the present invention.

FIG. 2 is a block diagram showing the architecture of the system, according to an exemplary embodiment of the present invention.

FIG. 3 illustrates a registration screen of an interface, according to an exemplary embodiment of the present invention.

FIG. 4 illustrates a login screen of the interface, according to an exemplary embodiment of the present invention.

FIG. 5 shows a screen of an interface, according to an exemplary embodiment of the present invention.

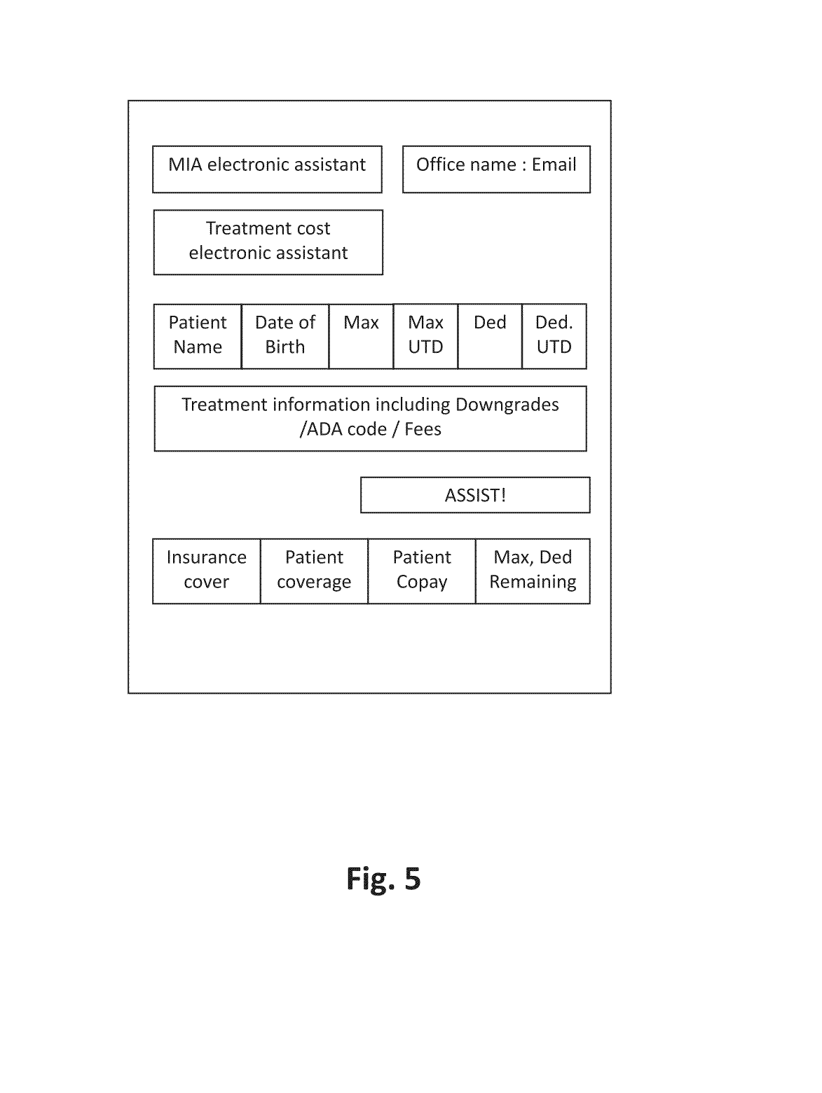

FIG. 6 shows an insurance providers screen of the interface, according to an exemplary embodiment of the present invention.

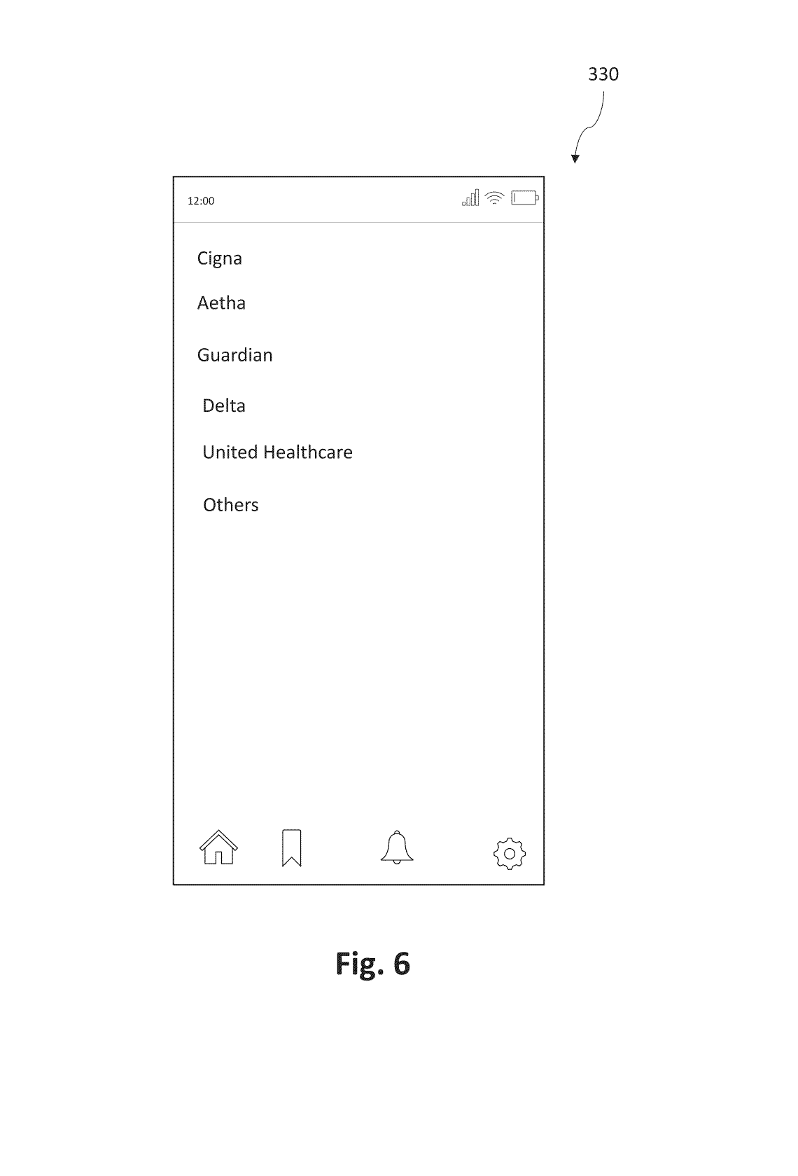

FIG. 7 illustrates a patient information screen of the interface, according to an exemplary embodiment of the present invention.

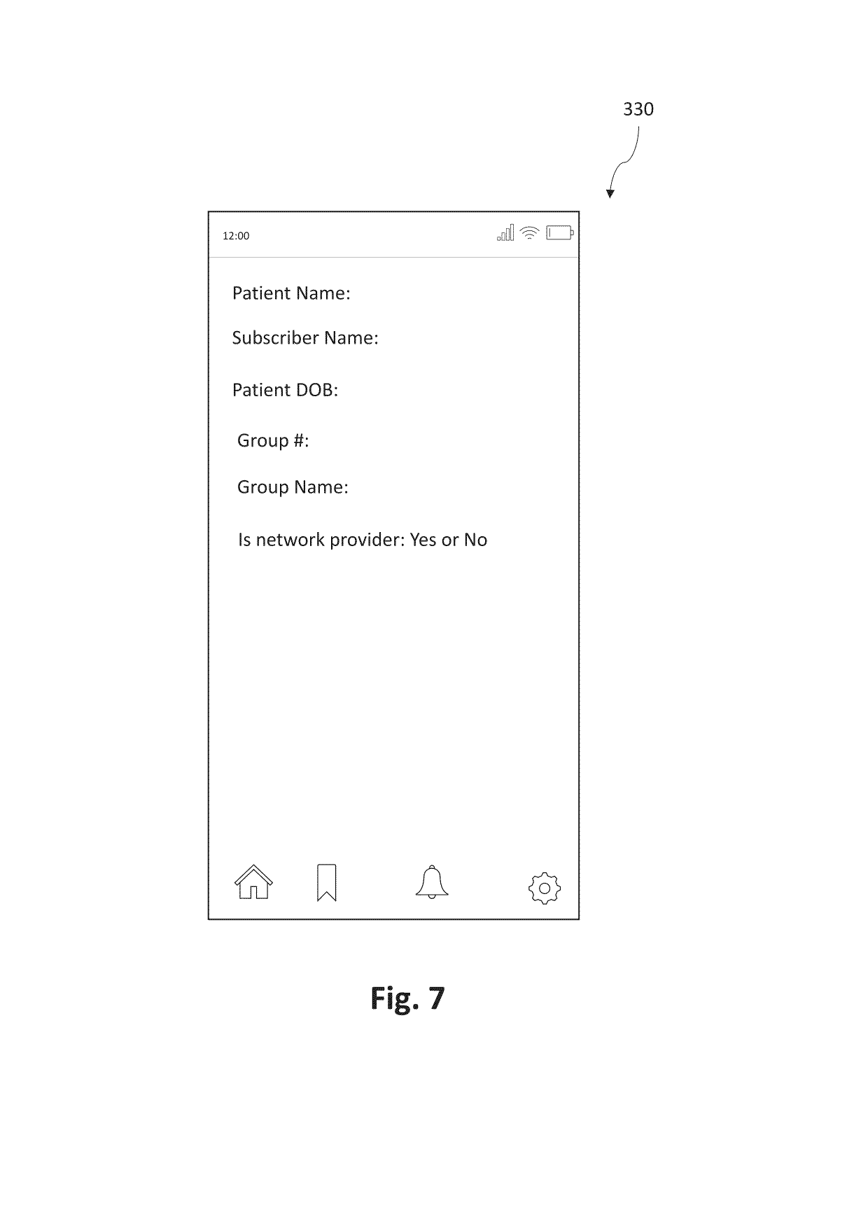

FIG. 8 shows insurance codes screen of the interface, according to an exemplary embodiment of the present invention.

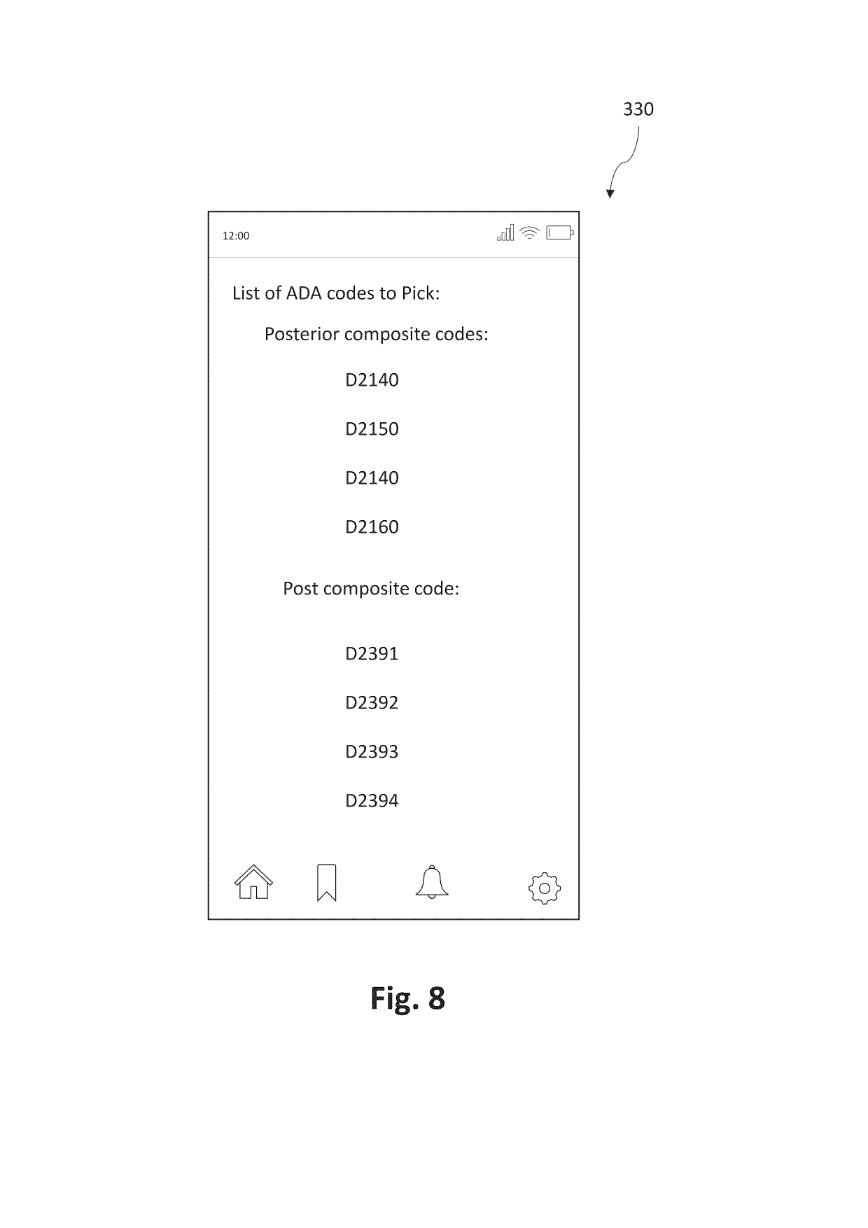

### DETAILED DESCRIPTION

The subject matter of the present invention will now be described more fully with reference to the accompanying drawings, which form a part of this disclosure and illustrate

specific exemplary embodiments. However, it should be understood that the subject matter may be embodied in various forms and is not limited to the specific embodiments set forth herein. Rather, these embodiments are provided by way of example to convey the scope of the invention. It is intended that the claims encompass a broad range of subject matter, including methods, devices, components, and systems. Accordingly, the following detailed description is not intended to be taken in a limiting sense.

As used herein, the term "exemplary" is intended to mean “serving as an example, instance, or illustration.” Any embodiment described as "exemplary" should not be construed as preferred or more advantageous over other embodiments. Similarly, the expression "embodiments of the present invention" does not imply that all embodiments must include all features, advantages, or modes of operation described.

The terminology used herein is for the purpose of describing particular embodiments only and is not intended to be limiting. As used herein, the singular forms “a,” “an,” and “the” include plural references unless the context clearly dictates otherwise. Furthermore, the terms “comprises,” “comprising,” “includes,” and/or “including” specify the presence of stated features, integers, steps, operations, elements, or components, but do not preclude the presence or addition of one or more other features, integers, steps, operations, elements, components, and/or groups thereof.

The following detailed description sets forth the best currently contemplated modes for carrying out exemplary embodiments of the invention. This description is not intended to be limiting, but rather to illustrate the general principles of the invention. The claims of any issued patent will define the scope of the invention.

The present invention pertains to a system and method configured to determine, in real time, data related to the costs and contributions associated with healthcare services to be made by a patient, healthcare facility, and insurance provider. The system accounts for multiple factors that may directly or indirectly influence the overall cost of healthcare services.

As used herein, the term “healthcare services” broadly refers to any form of medical treatment, including pre-treatment, treatment, and post-treatment procedures. The term further encompasses dental, optical, and preventive healthcare. The costs associated with such healthcare services include, but are not limited to, hospitalization charges, pre- and post-hospitalization expenses, physician consultations, surgical procedures, diagnostic tests, ambulance fees, and day-care treatments.

The term “insurance provider” as used herein refers to any entity that offers healthcare or medical insurance coverage to individuals. The insurance provider typically enters into contractual agreements with healthcare providers, which may define specific insurance codes for medical and dental procedures along with corresponding reimbursement rates. Similarly, the insurance provider enters into agreements with individuals or groups of individuals, referred to as insurance policies. These policies define the terms and conditions governing coverage, including deductibles, co-payments, coverage percentages, and applicable downgrades for specific procedures. The healthcare provider typically refers to institutions and/or people that provide healthcare services, and this includes clinics and hospitals.

The disclosed system is configured to receive and process both the contracts between insurance providers and healthcare providers, as well as the policies held by patients. By integrating these data sources, the system can determine accurate and dynamic cost estimates that reflect the true financial obligations of all involved parties.

In certain implementations, the disclosed system and method allow patients to obtain near real-time cost estimates for healthcare services with high accuracy relative to the actual final cost. A user can input or select a desired healthcare or dental procedure through a user device, such as a smartphone, tablet, or computer, and receive an immediate estimate based on their specific insurance parameters. The disclosed system eliminates the need for manual calculation, human intervention, or guesswork. It is designed to be patient-centric, providing highly reliable and transparent cost estimates that empower patients to make informed decisions quickly and confidently regarding their healthcare options.

The disclosed system can provide patients with instant and reliable answers to common questions regarding the costs of their medical or dental procedures. Patients no longer need to delay urgent or necessary care due to uncertainty about affordability. Instead, the disclosed system enables patients to determine, within seconds, the estimated total cost of each procedure and the corresponding contribution from their insurance provider.

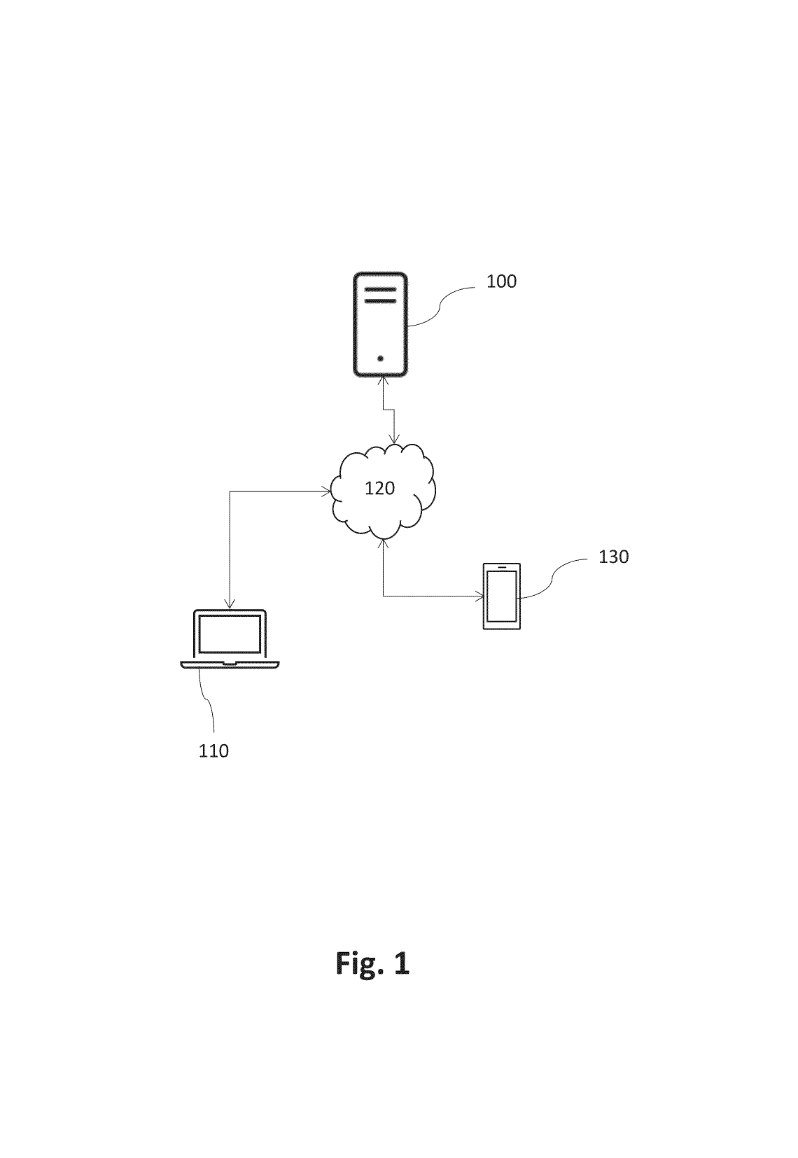

Referring now to FIG. 1, there is illustrated a block diagram showing an exemplary environment of the disclosed system 100. The system 100 is communicatively coupled to a user device 110 through a network 120. The user device 110 may be any computing device capable of executing instructions stored in memory via a processor. The device may include an input module for receiving user inputs, which can be provided through a touch display, keyboard, mouse, stylus, touchpad, voice interface, or other suitable input means. The user device may further include a display module for presenting information to the user, such as a graphical user interface (GUI) displaying estimated costs, insurance coverage breakdowns, and related data. The user device may also include network circuitry configured to establish wired or wireless communication with the network 120. Examples of the user device include, but are not limited to, a smartphone, desktop computer, laptop, tablet, or workstation.

The network 120 can be any type of communication network known in the art, including a wired network, a wireless network, or a combination thereof. Examples of such communication networks include, but are not limited to, a local area network (LAN), a wide area network (WAN), a wireless wide area network (WWAN), a wireless local area network (WLAN), a metropolitan area network (MAN), a wireless MAN, a cellular data network, a cellular voice network, or the Internet.

For illustration purposes, FIG. 1 depicts a single network connecting multiple user devices; however, those skilled in the art will recognize that multiple user devices may connect to the disclosed system through different networks. Likewise, a single user device may establish communication with the system through multiple networks simultaneously. For example, a user device may connect through both a LAN and the Internet to ensure stable and secure communication with the system 100.

The system 100 can also be operatively connected to one or more insurance provider servers 130 to retrieve and process insurance-related data for registered users. The insurance provider entities may upload or otherwise make available contractual details with various healthcare service providers, as well as policy information for individual users. These details can include payment terms and conditions, coverage parameters, co-pay structures, downgrades, exclusions, and any special provisions that affect the calculation of final costs for healthcare services.

In certain embodiments, all types of data relevant to generating accurate cost estimates may be incorporated into the insurance provider servers 130 by the insurance entities. Such data may be made available either directly by the insurance providers or indirectly through authorized third-party data aggregators that collect and supply this information to the disclosed system.

It should be understood that FIG. 1 illustrates the system connecting to a representative insurance provider server for exemplary purposes only. A person skilled in the art will recognize that the system may access insurance data through any suitable data exchange mechanism without departing from the scope of the present invention.

Because insurance data is often subject to frequent updates, the system may be configured to monitor and synchronize such updates either continuously or at predetermined intervals. This ensures that the user’s cost estimates remain current and reliable. In instances where insurance data changes shortly after a user has received a cost estimate for a particular healthcare service, the system can automatically notify the user that updated cost information is available, thereby maintaining transparency and accuracy.

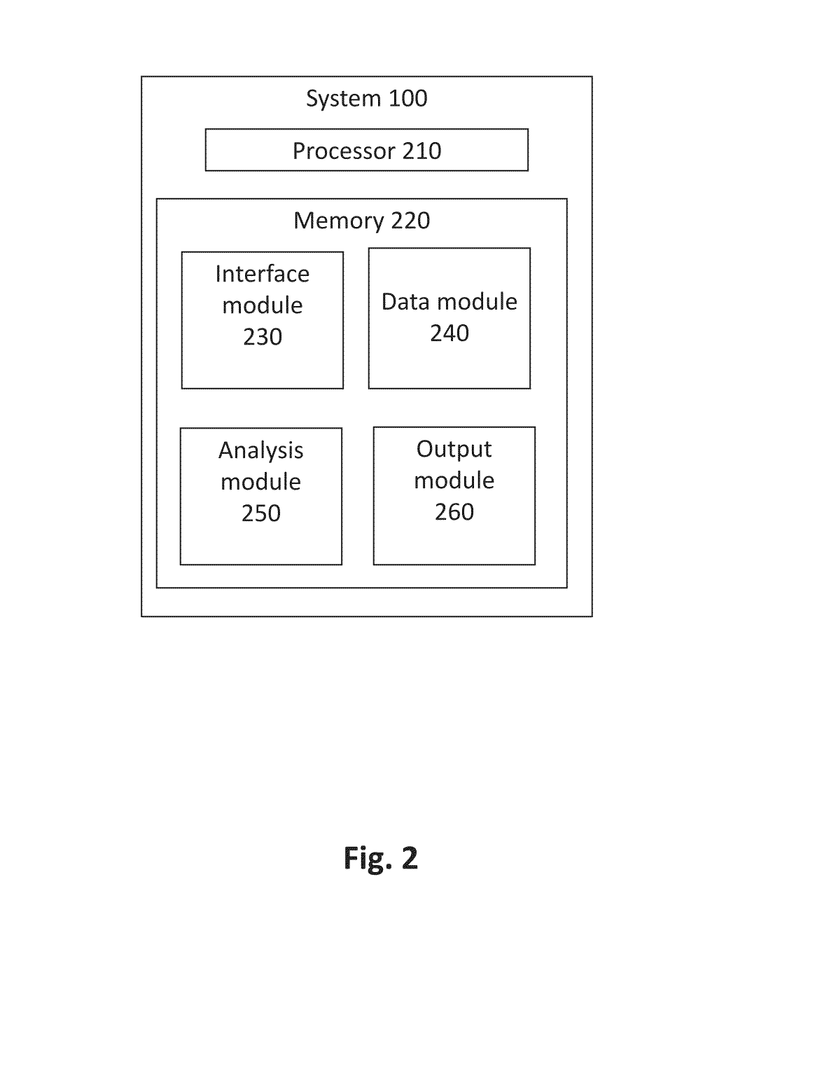

Referring now to FIG. 2, the disclosed system 100 includes a processor 210 and a memory 220 operably coupled to the processor. The processor 210 may comprise any logic circuitry, microcontroller, central processing unit (CPU), or equivalent processing component configured to execute instructions and process data stored in the memory. The memory 220 may include one or more memory devices, such as volatile or non-volatile storage media, capable of storing program instructions, data structures, and operational parameters, with direct access capability by the processor. The memory 220 can store executable modules and associated data configured to implement the functionality of the present invention. Upon execution by the processor, these modules enable one or more steps of the disclosed system and method for managing and estimating healthcare costs.

In one embodiment, the memory 220 may include an interface module 230, which, upon execution by the processor, generates and renders a user interface on the user device to facilitate user interaction with the system; a data module 240, which, upon execution, retrieves and aggregates data from multiple sources—including insurance provider servers, healthcare provider databases, and policy repositories—relevant to cost estimation of healthcare services; an analysis module 250, which processes user inputs received through the interface in combination with stored data to determine accurate and dynamic cost estimates for the requested healthcare or dental services; and an output module 260, which generates and presents the calculated cost estimates through the user interface on the user device, and provides contextual responses to user queries regarding the estimates, coverage details, and payment breakdowns.

As used herein, the term “module” refers to software, program code, a set of executable rules or instructions, or equivalent logic written in one or more computer-readable languages, including graphical or visual programming languages. Upon execution by the processor, a module performs one or more steps of the disclosed methodology. Although certain operations are described herein as being executed sequentially, it should be understood that, in some embodiments, the operations may be performed in parallel, concurrently, or within a distributed computing environment. The program code may be stored locally or remotely and may execute on single-processor or multi-processor machines.

Additionally, in some implementations, the order of operations may be rearranged or modified without departing from the spirit or scope of the disclosed subject matter

The disclosed system can be implemented using one or more servers, including cloud-based servers. These servers may be located in a single physical location or geographically distributed across multiple regions for redundancy, scalability, or performance optimization. In certain embodiments, one or more steps of the disclosed methodology may also be performed directly on user devices, enabling distributed processing without deviating from the intended functionality of the present invention.

The interface module 230 provides an interactive interface that allows users to communicate with the disclosed system through a user device. The interface may comprise a series of dynamic screens or pages that present information to the user, receive input from the user, and facilitate the execution of one or more steps of the disclosed methodology. The interface can be implemented as application software (app) installed on the user device.

The application software may be developed for various mobile operating platforms, including but not limited to Android™, iOS, or other known mobile systems, and can be distributed via recognized platforms such as Google Play™ or the Apple App Store™. Additionally, a web-based interface may be provided via the World Wide Web for browser access, and desktop-compatible versions may be developed for operating systems such as Windows™, Linux, and macOS.

The user interface allows bi-directional communication, wherein system 100 can present real-time cost information, coverage breakdowns, and related data to the user, and can also receive user inputs such as service selections, policy details, or queries regarding healthcare cost estimates.

A user can register with the disclosed system to obtain secure access to its features and services. A user module may be configured to receive basic identifying information from the individual, such as name, contact details, email address, and other relevant credentials. Upon successful registration, the user module can generate login credentials for secure access to the disclosed system.

The login credentials may include at least a username and a password, where the password may be an alphanumeric code, biometric identifier (such as a fingerprint or facial recognition), or security token. In certain embodiments, the system may support multiple login options and multi-factor authentication (MFA) to enhance user security.

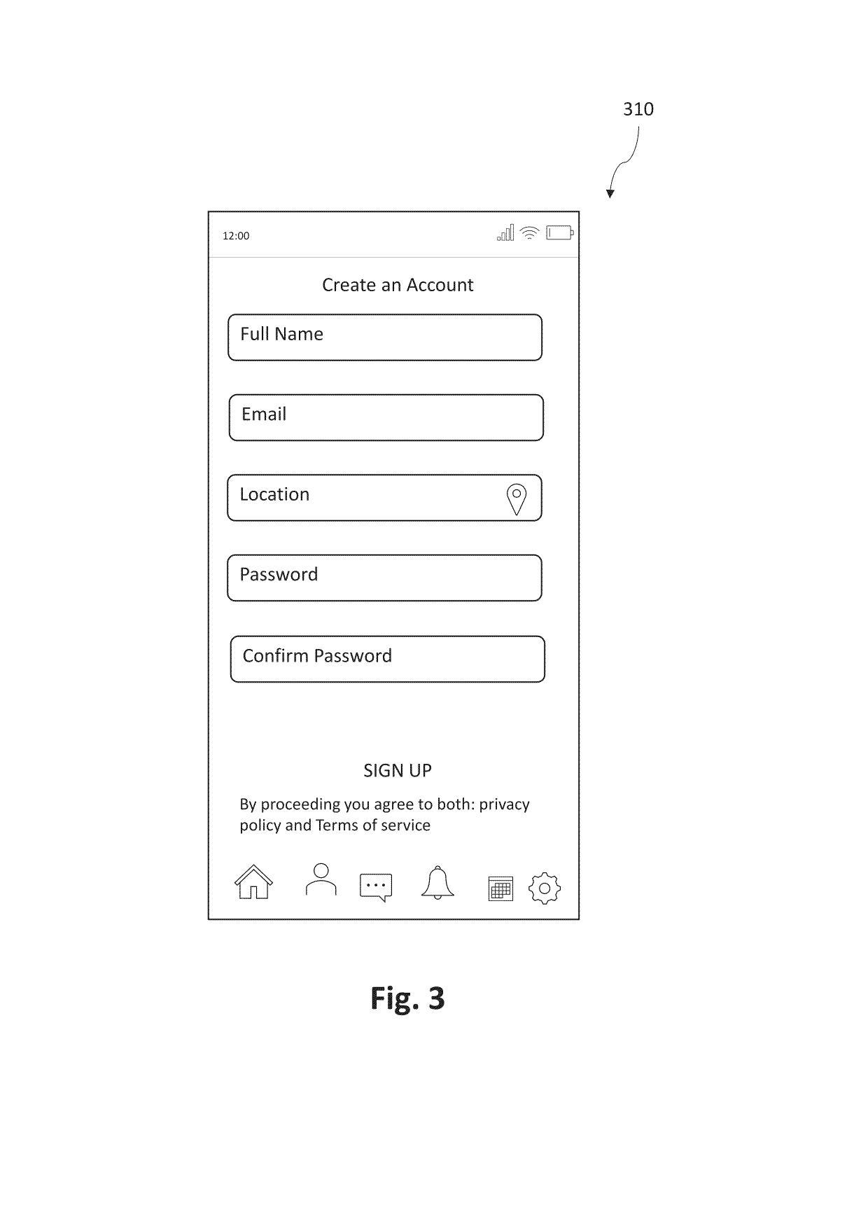

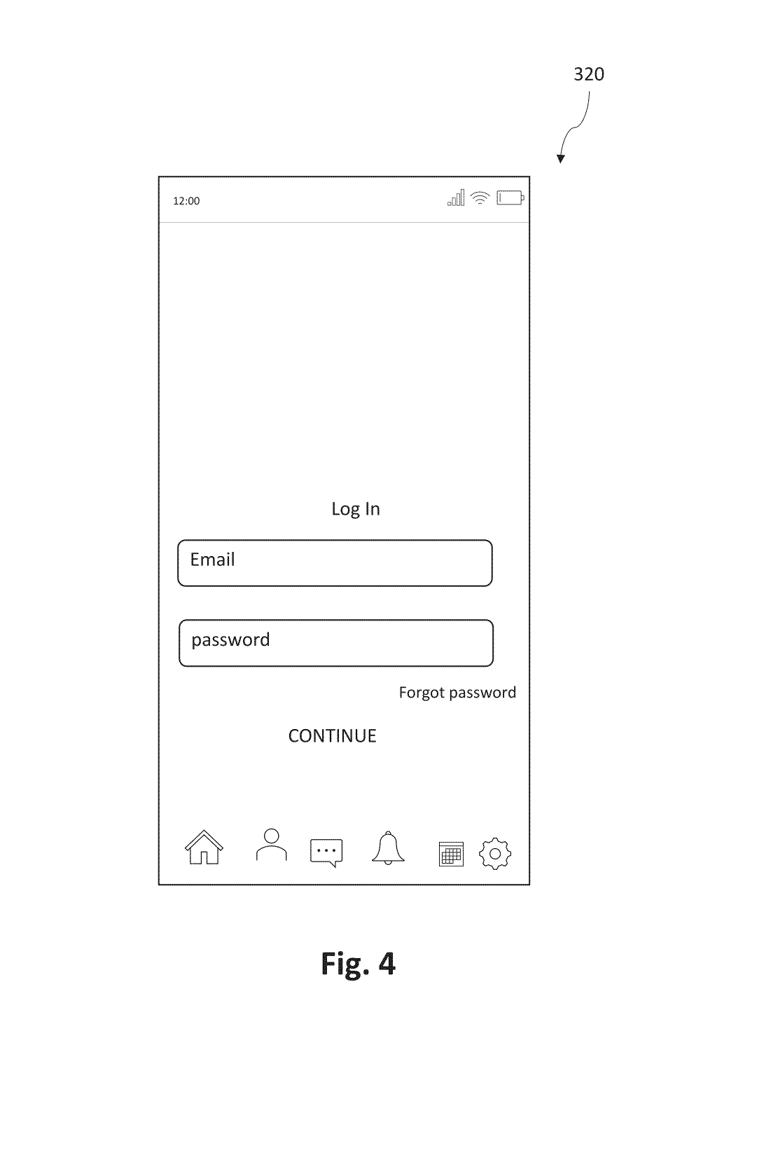

The user can be presented with a login interface rendered on the user device to access the system. As illustrated in FIG. 3, an exemplary registration screen 300 may include a form-like input interface for receiving basic user information, while FIG. 4 illustrates a login screen 400 that enables the user to securely access the system using the generated credentials.

Upon registration, the system can generate a user profile and store it in a suitable database. The databases, including their structures and operations, may be any type known in the art. In certain embodiments, blockchain-based databases or other secure distributed ledger technologies may be employed to enhance transparency, immutability, and data security.

The created user profile can be modified or updated by the user at any time. The system may present an editable form interface that allows users to change or add information, after which the system automatically updates the stored profile in the database. Preferably, the user profile may include the user’s name, contact details, social security number or other identification numbers, and relevant insurance policy details.

The data module 240 can retrieve and aggregate data from various sources, including insurance providers, healthcare service providers, and other authorized data repositories. Such data may be stored in diverse formats and across multiple databases. The data module may include predefined templates or APIs to facilitate structured and efficient data retrieval.

In preferred embodiments, the process of importing data from external servers and databases is automated, thereby improving speed, accuracy, and consistency. This automation allows users to receive real-time or near real-time updates, ensuring that cost estimates are generated quickly and reflect the most current available data.

In certain implementations, the interface module can render a dedicated interface for an insurance service provider device to enable authorized insurance entities to input, verify, or update relevant information directly into the disclosed system. The insurance service provider may manually feed or upload data through this interface, which can include plan structures, premium updates, or reimbursement policies. Additionally, the disclosed system can assist in the processing of healthcare claims, estimation of hospitalization expenses, and treatment cost evaluations, thereby streamlining administrative coordination among insurers and healthcare providers.

In certain implementations, the disclosed system can also provide an interface for use on healthcare provider devices, such as devices operated by healthcare staff, billing or accounts teams, and medical practitioners. This interface can guide healthcare providers in advising patients regarding potential treatment costs. Through this interface, the system may also track actual treatment expenses as they occur. Any variations in cost estimates arising from ongoing treatment changes can be automatically determined by the system and communicated to the patient. Thus, the disclosed system facilitates real-time cost tracking and reconciliation between the patient, healthcare provider, and insurance provider. The system can compute co-payments, deductibles, and remaining balances in near real time, thereby significantly reducing manual computation time and administrative delays.

In operation, a user, such as a patient or an authorized representative, may initiate a cost estimation request through the user interface. Upon receiving such a request, the data module automatically imports relevant data from multiple authenticated sources, including insurance provider databases, healthcare service provider databases, and patient profile databases.

For insurance provider databases, the system can retrieve plan-specific details such as policy type, deductible amounts, co-payment structures, downgrade rules, contribution percentages, and annual maximum limits.

For healthcare provider databases, the system can retrieve procedure codes, provider-specific pay scales, and fee schedules associated with each treatment or service.

For patient profile databases, the system can access demographic data, policy details, and prior claim histories to accurately map applicable deductibles, co-insurance levels, and coverage limits.

In certain implementations, the data import process from different databases can be fully automated and performed in real time using secure APIs or standardized data exchange protocols such as HL7 or FHIR. The system continuously synchronizes with external databases to ensure that the most current insurance policy and pricing information are reflected in cost estimation calculations, eliminating the need for manual updates.

Once all relevant data are imported, the analysis module executes a multi-tiered computational process to generate the cost estimate. The process includes: Identification of the correct insurance provider based on parameters such as provider ID, geographic region, and applicable pay scale; Parsing of insurance information to apply correct plan details, coverage limits, and deductible requirements; Alignment of provider rates with the insurer’s contribution structure to ensure accuracy; Evaluation of procedure codes (e.g., CPT, CDT, ICD) for each treatment or diagnostic service, retrieving both the provider’s contracted fee and the insurance company’s allowable reimbursement amount; and Application of plan rules and adjustments, including remaining deductibles, annual maximums, downgrade or alternate benefit provisions, and co-insurance percentages.

The system analyzes all contributing factors to determine a range of cost estimates, comprising a lowest possible cost and a highest possible cost. The highest possible cost may be based on the lowest insurer contribution and potential out-of-network surcharges or uncovered procedure downgrades. Conversely, the lowest possible cost may be determined based on the highest insurer contribution and lowest in-network provider charges. This range provides both patient and provider with realistic financial expectations, ensuring that actual billed costs remain within predicted bounds and minimizing billing discrepancies at the time of payment.

The output module can present the cost estimation in both a summary view and a detailed breakdown, depending on user preference. The estimation process may be explainable, allowing the user to view the underlying data and calculation logic that contributed to the estimate. This transparency fosters trust and enables users to make informed healthcare decisions.

The disclosed system may further employ artificial intelligence (AI) to enhance the speed, accuracy, and adaptability of cost estimation. The AI model can be trained on historical claim data and payment feedback from multiple insurance and healthcare service providers. Over time, the AI engine refines its prediction accuracy by comparing estimated costs against actual incurred costs, thereby narrowing the cost estimate range. The continuous learning capability enables the system to adapt to changing insurance policies, provider fee structures, and claim handling practices. Additionally, the system may monitor ongoing treatments and associated costs, using such data to recalibrate future estimates via statistical or AI-driven predictive models.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A system for determining real-time healthcare cost estimates, comprising:
a processor; and
a memory operably coupled to the processor, the memory storing a set of instructions which upon execution by the processor causes the processor to:
render an interface on a user device for receiving user input and presenting cost estimation results;
automatically retrieve healthcare-related data from a plurality of authenticated data sources including insurance provider databases, healthcare service provider databases, and patient profile databases;
process, through an analysis module, the retrieved data to determine estimated costs for a healthcare service based on parameters comprising policy-specific rules, provider fee schedules, procedure codes, co-pays, deductibles, downgrades, contribution percentages, and annual maximums; and
display, on the user interface, a cost estimate range including a lowest and highest possible cost and a detailed breakdown of costs and contributions.

2. The system of claim 1, wherein the data is retrieved through secure communication protocols comprising HL7, FHIR, or application programming interfaces (APIs).

3. The system of claim 1, wherein the analysis module identifies a correct insurance provider using parameters comprising provider ID, geographic region, and pay scale.

4. The system of claim 3, wherein the analysis module applies policy-specific factors including remaining deductible, annual maximums, downgrades, alternate benefit provisions, and co-insurance percentages.

5. The system of claim 1, wherein explainable cost estimates are presented by displaying underlying factors contributing to the estimated cost.

6. The system of claim 1, wherein the system further comprises an artificial intelligence (AI) engine configured to learn from historical claim data and actual treatment payments to improve future cost estimation accuracy.

7. The system of claim 6, wherein the AI engine continuously updates a predictive model based on feedback from insurance provider contributions and healthcare provider invoices.

8. The system of claim 1, wherein the system continuously synchronizes with external databases to ensure updated insurance or pricing information is reflected in the cost estimation.

9. The system of claim 8, wherein the processor sends a notification to the user when an updated insurance dataset changes a previously calculated cost estimate.

10. A computer-implemented method for determining healthcare cost estimates, comprising the steps of:
receiving, through a user interface, a healthcare service cost estimation request from a user;
automatically importing data from insurance provider databases, healthcare service provider databases, and patient profile databases;
identifying a corresponding insurance provider and applying policy parameters including co-pays, deductibles, and contribution percentages;
retrieving procedure codes and associated provider fee schedules;
calculating a cost estimate range based on highest and lowest insurer contributions and provider rates; and
presenting, through the user interface, the cost estimate range and an itemized cost breakdown.

11. The method of claim 10, further comprising continuously synchronizing with external databases through secure APIs to maintain current insurance and provider pricing data.

12. The method of claim 10, further comprising training an AI model using historical claim data to refine cost estimation accuracy over time.

13. The method of claim 12, wherein the AI model adjusts predictive parameters based on discrepancies between estimated and actual incurred costs.

14. The method of claim 11, further comprising sending a notification to the user when a change in insurance or provider data alters a previously determined cost estimate.
